(19) United States
(12) Patent Application Publication
(10) Pub. No.: US 2025/0157626 A1
(43) Pub. Date: May 15, 2025

(54) METHOD FOR AUTOMATICALLY SCREENING NEUROFEEDBACK TRAINING PROTOCOLS AND RECOMMENDING RESULTS

(71) Applicant: ExeBrain Co., Ltd., Taipei City (TW)

(72) Inventors: Shu-Chun Kuan, Taipei City (TW); Jason Chun-Cheng Lin, Taipei City (TW)

(21) Appl. No.: 18/827,971

(22) Filed: Sep. 9, 2024

(30) Foreign Application Priority Data
Nov. 9, 2023 (TW) 112143313

(51) Int. Cl. G16H 20/70; G06F 3/01; G16H 10/60; G16H 40/67

(52) U.S. Cl. CPC G16H 20/70; G06F 3/015; G16H 10/60; G16H 40/67

(57) ABSTRACT

A method for automatically screening neurofeedback training protocols and recommending results is disclosed. The steps of the method include capturing a biological data of a subject by a brainwave collection device, wherein the brainwave includes a home brainwave collection device; transmitting the biological data to a brainwave database of a remote cloud system through a network; converting the biological data into a corresponding training parameter recommendation by the brainwave database; and remotely feeding back the training parameter recommendation to a neurofeedback cognitive training module for the subject to perform cognitive training. Through this, a software interface is provided to provide recommended training parameters, arrangement of strengths and weaknesses of the brain area network, digital therapy recommended training protocols and related health education, thereby achieving the effect of remote feedback and home brain training to improve cognitive abilities.

### CROSS-REFERENCE TO RELATED APPLICATIONS

This application claims the priority of Taiwanese patent application No. 112143313, filed on Nov. 9, 2023, which is incorporated herewith by reference.

### PRIOR ART

Prior art biofeedback training mainly uses wireless devices at the input terminal, for example, using a pair of electrode patches to compare brainwave changes before and after training in three areas of the parietal lobe, using a pair of electrode patches to detect the impact of neurophysiological feedback on sensorimotor rhythm (SMR), or collecting physiological signals and upload physiological data to the cloud platform for analysis through wired or wireless transmission modules. Individual users need to open the APP or related applications to retrospectively read the physiological device during sleep periods. However, prior art technology usually prevents subjects from immediately obtaining physiologically relevant information such as brainwaves or heartbeat variations, and they need to wait for hours to days for interpretation.

At the same time, although the prior art intelligent bed health management system also collects physiological signals, what is collected is the physiological signals of individuals while sleeping in bed. Physiological signals are uploaded to the cloud platform for analysis through wired or wireless transmission modules. The individual needs to open the relevant application to retrospectively read the physiological device during sleep. The disadvantage is that individual physiological signals cannot be immediately processed and fed back to the subject after being transmitted.

In addition, although there is a neurofeedback mechanism for functional magnetic resonance imaging (fMRI), the equipment for magnetic resonance imaging is quite expensive and is mostly installed in medical institutions. The signal collection to imaging process takes more than 30 minutes, and the calculation of the feedback mechanism also takes more than 10 minutes. It is impossible to achieve remote home configuration and real-time (within 1 minute) analysis feedback. Moreover, even if feedback is obtained, the subject cannot immediately know how to perform training. Instead, the information must be provided to professionals (such as doctors), and training plans and related health education will be provided after professional judgment. It is impossible to achieve remote and immediate feedback.

### BACKGROUND OF THE INVENTION

The present invention relates to the technical field of neurofeedback, in particular to a method for automatically screening neurofeedback training protocols and recommending results.

### SUMMARY OF THE INVENTION

A primary objective of the present invention is to provide a method for automatically screening neurofeedback training protocols and recommending results. After capturing the subject's brainwave data, a software interface is used to provide recommended training parameters, arrangement of strengths and weaknesses of the brain area network, digital therapy recommended training programs and related health education, so as to achieve the effect of remote feedback and at-home brain training to improve cognitive abilities.

A method for automatically screening neurofeedback training protocols and recommending results is disclosed. The method comprises capturing a biological data of a subject by a brainwave collection device, wherein the brainwave includes a home brainwave collection device; transmitting the biological data to a brainwave database of a remote cloud system through a network; converting the biological data into a corresponding training parameter recommendation by the brainwave database; and remotely feeding back the training parameter recommendation to a neurofeedback cognitive training module for the subject to perform cognitive training.

In some embodiments, the home brainwave collection device is an electroencephalography (EEG) cap or a heart rate variability cap (HRV Cap).

In some embodiments, the network is an ad-hoc Network.

In some embodiments, the biological data is a scalp electroencephalography (EEG) signals from one or more channels, wherein the training parameter recommendation is mainly converted into a standard score through at least one channel of the scalp electroencephalography (EEG) signals (e.g. 19-channel brainwave data) according to the calculation, and then presented in an order list according to a deviation mean.

In some embodiments, the brainwave data includes amplitudes, frequencies, sites and pattern characteristics.

In some embodiments, after the step of remotely feeding back the training parameter recommendation to a neurofeedback cognitive training module for the subject to perform cognitive training, the method further comprises providing an effect suggestion based on a result of the cognitive training, and transmitting the result of the cognitive training through the network back to the remote cloud system.

In some embodiments, the neurofeedback cognitive training module includes a desktop computer, a notebook computer and/or a smart mobile device.

In some embodiments, the training parameters are recommended to include a low-resolution electromagnetic tomography (LORETA) brain area/network training and a surface brainwave training.

In some embodiments, a brain activity area is predicted back through the brainwave characteristics as the recommended training parameters based on the sites of the one or more channels.

In some embodiments, the step of converting the biological data into a corresponding training parameter recommendation by the brainwave database further comprises recommending to refer to a priority order of execution of training after comparing surface brainwaves and brain area network according to a norm through a training protocol recommendation after the calculation; and remotely feeding back to a neurofeedback cognitive training module based on the training parameter recommendation for the subject to perform a cognitive training step of neurophysiological feedback, and then comparing the norm to evaluate whether brainwaves or brain areas are approaching balance to evaluate effectiveness recommendations.

### BRIEF DESCRIPTION OF DRAWINGS

Aspects of the present invention are best understood from the following detailed description when read with the accompanying figures. It is noted that, in accordance with the standard practice in the industry, various features are not drawn to scale. In fact, the dimensions of the various features may be increased or reduced for clarity of discussion.

FIG. 1 is a flowchart of a method for automatically screening neurofeedback training protocols and recommending results in accordance with embodiments of the present invention.

FIG. 2 is a schematic view of a home brainwave collection device used in the method for automatically screening neurofeedback training protocols and recommending results in accordance with embodiments of the present invention.

FIG. 3 is a side view of a home brainwave collection device used in the method for automatically screening neurofeedback training protocols and recommending results in accordance with embodiments of the present invention.

FIG. 4 is a schematic view of brainwave feature analysis of the method for automatically screening neurofeedback training protocols and recommending results in accordance with embodiments of the present invention.

FIG. 5 is a block diagram of the method for automatically screening neurofeedback training protocols and recommending results in accordance with embodiments of the present invention.

### DETAILED DESCRIPTION OF THE INVENTION

It will be appreciated that, although specific embodiments of the present invention are described herein for purposes of illustration, various modifications may be made without departing from the spirit and scope of the present invention.

In the following description, certain specific details are set forth in order to provide a thorough understanding of various aspects of the disclosed subject matter. However, the disclosed subject matter may be practiced without these specific details. In some instances, well-known structures and methods of power delivery comprising embodiments of the subject matter disclosed herein have not been described in detail to avoid obscuring the descriptions of other aspects of the present invention.

Unless the context requires otherwise, throughout the specification and claims that follow, the word “comprise,” “have,” “include,” and variations thereof, such as “comprises,” “comprising,” “having,” “including” are to be construed in an open, inclusive sense, that is, as “including, but not limited to.”

Reference throughout the specification to “one embodiment” or “an embodiment” means that a particular feature, structure, or characteristic described in connection with the embodiment is included in at least one embodiment. Thus, the appearance of the phrases “in one embodiment” or “in an embodiment” in various places throughout the specification are not necessarily all referring to the same aspect. Furthermore, the particular features, structures, or characteristics may be combined in any suitable manner in one or more aspects of the present invention.

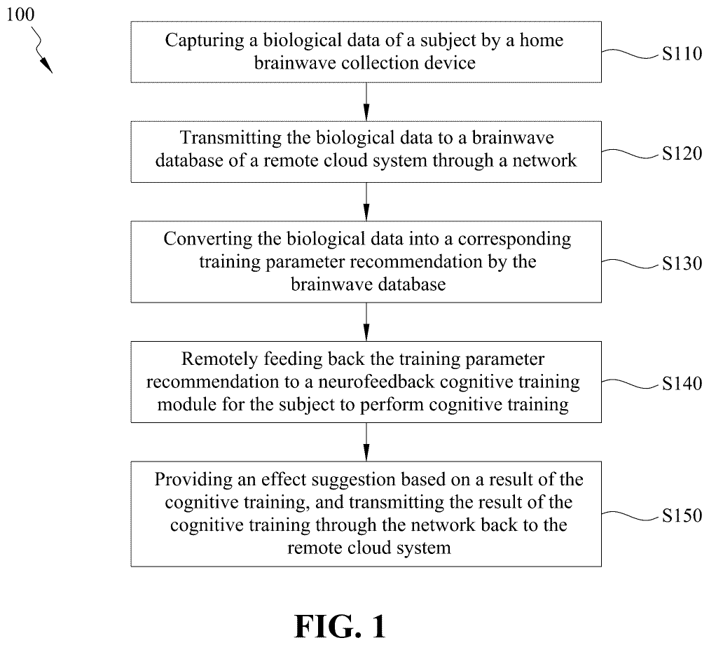

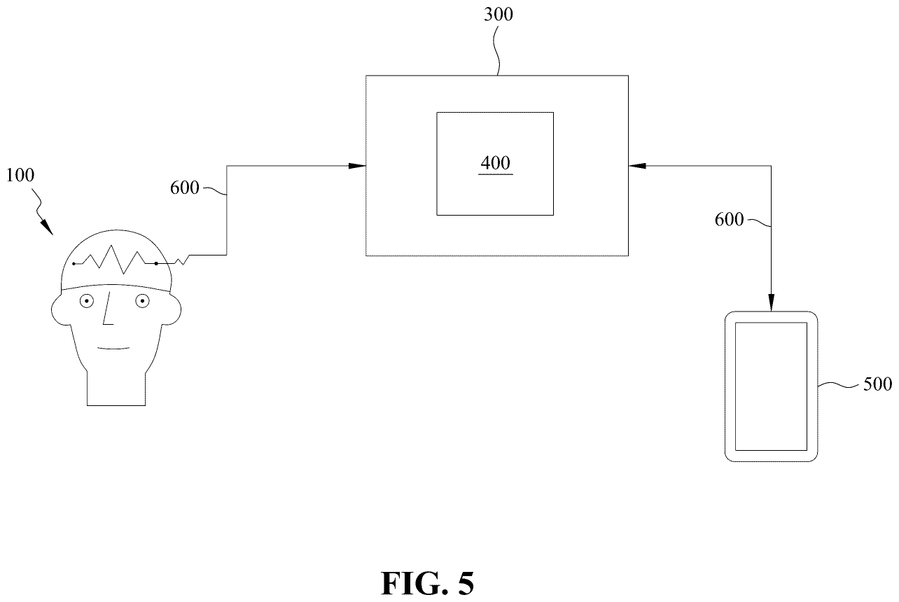

FIG. 1 is a flowchart of a method for automatically screening neurofeedback training protocols and recommending results in accordance with embodiments of the present invention. FIG. 5 is a block diagram of the method for automatically screening neurofeedback training protocols and recommending results in accordance with embodiments of the present invention. Please refer to FIGS. 1 and 5, the method S100 for automatically screening neurofeedback training protocols and recommending results includes capturing a biological data of a subject by a brainwave collection device, wherein the brainwave includes a home brainwave collection device 100 (step S100); transmitting the biological data to a brainwave database 400 of a remote cloud system 300 through a network 600 (step S120); converting the biological data into a corresponding training parameter recommendation by the brainwave database 400 (step S130); and remotely feeding back the training parameter recommendation to a neurofeedback cognitive training module 500 for the subject to perform cognitive training (step S140). In some embodiments, the network 600 may be an ad-hoc Network, but is not limited thereto. In some embodiments, the brainwave collection device can be a home brainwave collection device 100, but it is not limited thereto. Brainwave collection devices used in other fields are also included in the protection scope of the embodiments of the present invention.

In some embodiments, after the step S140 of remotely feeding back the training parameter recommendation to a neurofeedback cognitive training module for the subject to perform cognitive training, the method S100 further comprises providing an effect suggestion based on a result of the cognitive training, and transmitting the result of the cognitive training through the network back to the remote cloud system 300 (step S150).

In some embodiments, in step S103, it further comprises recommending to refer to a priority order of execution of training after comparing surface brainwaves and brain area network according to a norm through a training protocol recommendation after the calculation; and remotely feeding back to a neurofeedback cognitive training module based on the training parameter recommendation for the subject to perform a cognitive training step of neurophysiological feedback (step S140), and then comparing the norm to evaluate whether brainwaves or brain areas are approaching balance (approaching standard Z-score) to evaluate effectiveness recommendations.

In some embodiments, the training parameters are recommended to include a low-resolution electromagnetic tomography (LORETA) brain area/network training and a surface brainwave training.

In some embodiments, the neurofeedback cognitive training module 500 includes a desktop computer, a notebook computer and/or a smart mobile device, but is not limited thereto. Through this, subjects can perform “brain area training” or “brainwave training” through neurofeedback cognitive training module 500.

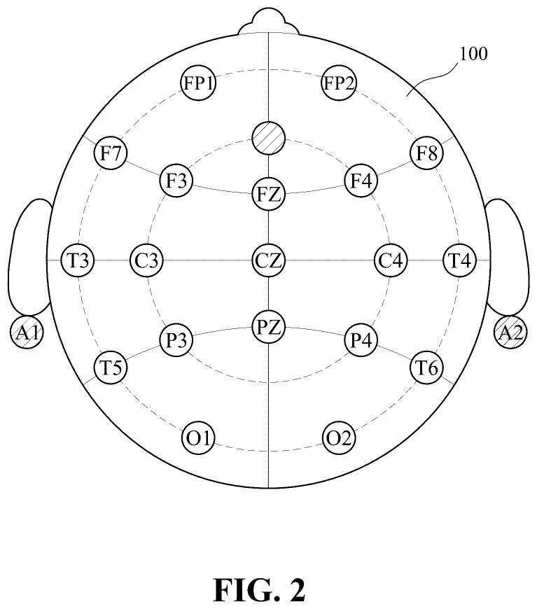

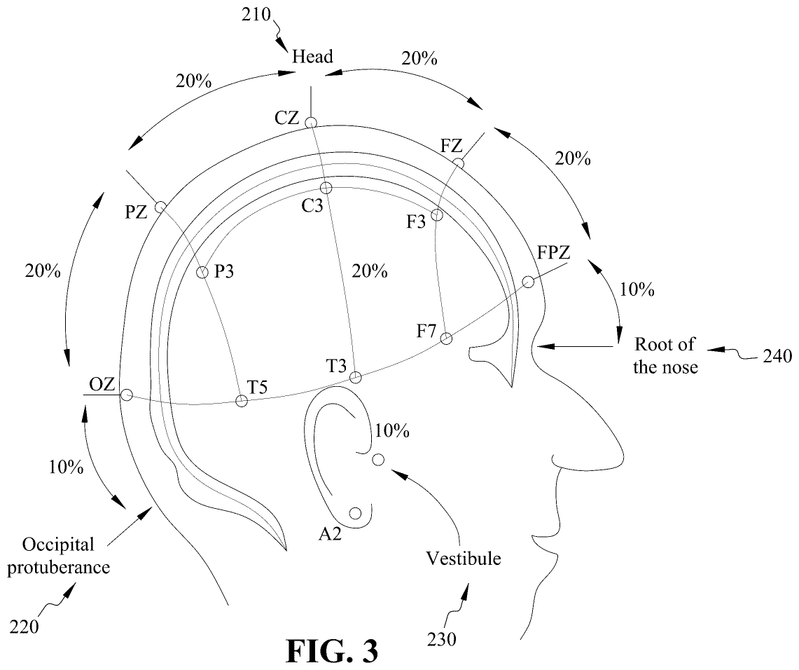

FIG. 2 is a schematic view of a home brainwave collection device used in the method for automatically screening neurofeedback training protocols and recommending results in accordance with embodiments of the present invention. FIG. 3 is a side view of a home brainwave collection device used in the method for automatically screening neurofeedback training protocols and recommending results in accordance with embodiments of the present invention. Please refer to FIGS. 2 and 3, the home brainwave collection device 100 used in the method S100 of the present invention for automatically screening neurofeedback training protocols and recommending results may be an electroencephalography (EEG) cap or a heart rate variability cap (HRV Cap), and FIGS. 2 and 3 use the EEG Cap as an example for illustration, but are not limited thereto.

Please refer to FIGS. 2 and 3, the home brainwave collection device 100 is worn in contact with the subject's head 210, Inion 220, vestibule 230 and the root of the nose 240, and the home brainwave collection device 100 includes electrodes C3, C4, P3, P4, O1, O2, FP1, FP2, FZ, CZ, PZ, T3, T4, T5, T6, F3, F4, F7, F8 and ear electrodes A1 and A2, that is, the brain activity areas are positioned through the combination of brainwave sites and brainwave patterns of the 19 channels of the above 19 electrodes.

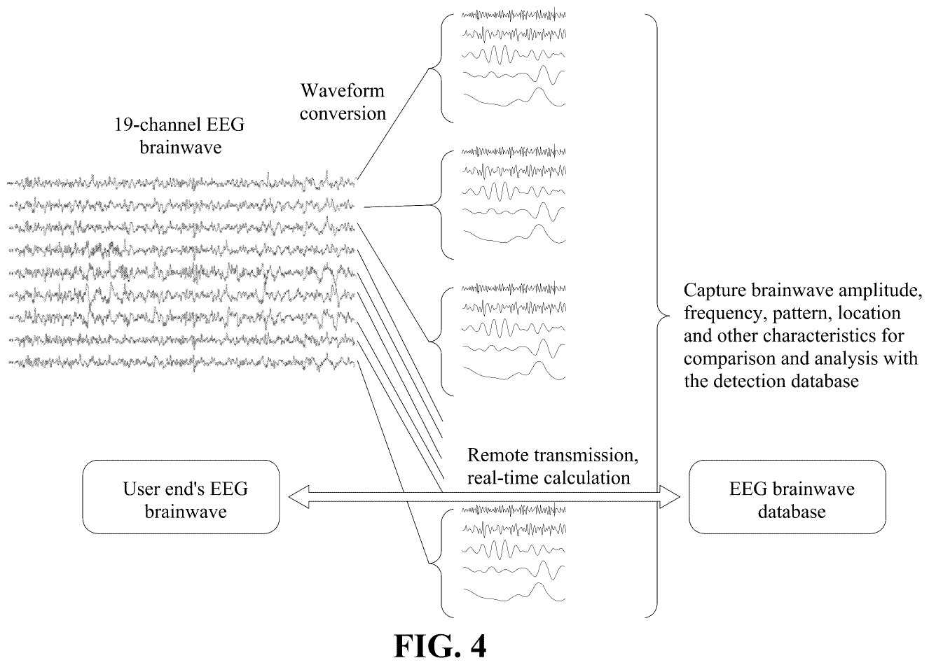

FIG. 4 is a schematic view of brainwave feature analysis of the method for automatically screening neurofeedback training protocols and recommending results in accordance with embodiments of the present invention. In some embodiments, brain activity areas are positioned based on the combination of 19-channel brainwave sites and brainwave patterns of the home brainwave collection device used above. Therefore, the biological data may be a 19-channel EGG (Electroencephalography) brainwave data. In some embodiments, the biological data is scalp electroencephalography (EEG) signals from one or more channels Therefore, it is recommended that the training parameters mainly use the at least one channel of the scalp electroencephalography (EEG) signals (e.g. 19-channel brainwave data), convert it into a standard score according to the calculation, and then present an order list according to the deviation mean. The brainwave data may include amplitude, frequency, site and pattern characteristics, but is not limited thereto. As shown in FIG. 4, The brainwave characteristics detected through the 19 channels (that is, the 19 electrodes FP1, FP2, F3, F4, F7, F8, FZ, T3, C3, CZ, C4, T4, T5, P3, PZ, P4, T6, O1, O2 in FIGS. 2 and 3) of the home brainwave collection device 100 include four characteristic parameters: vibration, frequency, brainwave site (sites of 19 electrodes FP1, FP2, F3, F4, F7, F8, FZ, T3, C3, CZ, C4, T4, T5, P3, PZ, P4, T6, O1, O2), and amplitude, frequency, shape, and position of the brainwave pattern. The four characteristic parameters constitute the brainwave database of different ethnic groups through. The sites of the 19 channels are basically positioning to predict brain activity areas backwards through brainwave characteristics, which can be used as parameters for the above comparison and training. In this embodiment, the brainwaves collected by the home brainwave collection device 100 may be compared with the brainwave patterns to obtain a basic score. As shown in FIG. 4, after comparison and analysis with the brainwave database 400, a benchmark point for the index score may be generated. This benchmark point is also the difference compared to the norm, in similar groups (same age, same education level, same gender, etc.).

For example, if the brainwave collected by a subject through the home brainwave collection device 100 is converted into a score of X, but the ideal score of the subject compared with the database should be Y, then during the neurofeedback training process, the goal is to reduce the difference between X and Y, and the subject will receive feedback messages when the difference is reduced to a certain ratio. After the subject accepts the feedback message, he or she can perform another brainwave pattern comparison. At this time, the brainwaves collected by the home brainwave collection device 100 can be converted into a new benchmark score X′. This X′ will also be compared with the database, and the new neurofeedback training goal is to shorten the difference between X′ and Y. Neurofeedback training is much like muscle training for the brain. If you want to exercise a certain muscle (biceps of the hand, six-pack muscles of the abdomen, thigh muscles of the legs, etc.), the muscle endurance before exercise is X, but the goal is to obtain Y strength, then you will gradually train specific muscle groups until X-Y gets closer and closer. For example, if you want to be able to lift a 30 kg (Y) dumbbell, but currently the user end can only lift 10 kg (X), if the user end can lift 15 kg, then feedback will be given (X-Y is shortened by a certain percentage). After exercising for a period of time and reassessing, the user end can lift a 20 kg (X′) dumbbell. At this time, the user end may need to lift a 25 kg dumbbell (X′-Y is shortened by a certain proportion) to get feedback. For example, if a subject with inattention wants to improve his concentration through brain training, he can analyze the collected brainwaves. If it is found that the frontal lobe area of the brain is overactivated compared with the norm, feedback can be used to gradually reduce the difference between X and Y.

Specifically, after analysis and conversion of the raw EEG signal, the frequency and amplitude of the brainwave signal recorded at each electrode site that changes with time serials may be obtained. The results of the analysis and conversion of the brainwaves recorded by a single electrode compared with the established brainwave normative database may be converted into a “standard score (Z score)” based on the parameters such as gender, age, and dominant hand corresponding to the database to obtain the relevant position of the brainwave activity. The calculation results in a value “a few standard deviations above or below the mean.” The normal distribution of Z scores is defined as a data distribution with a mean of 0 and a standard deviation of 1.

The standard Z score is calculated as: Z=(x−μ)/σ, where x is the value of the raw brainwave after analysis and conversion, μ is the average number of the brainwave normative database, and σ is the standard deviation of the normative database. The Z-score represents the distance between the raw brainwave value and the normative database mean, calculated in units of standard deviation. When the raw brainwave score is standardized and is lower than the average of the normative database, the Z-score is a negative number; when the raw score is standardized and higher than the average of the normative database, the Z-score is a positive number. After conversion of the brainwave parameters, if the brainwave parameters are lower than the allowable range, it is defined as “hypo-activity”; conversely, if it is higher than the allowable range, it is defined as “hyper-activity”.

The brainwave data obtained at each site are converted and compared with the brainwave normative data. That is to say, each site has a corresponding brainwave database norm, which is arranged in order according to the deviation of the standard score from the norm, which can correspond to the recommended training site for surface brainwave training. After comparing the site with the norm, if it is “hypo-activity”, it is by enhancing the characteristics of the brainwaves in the area (for example, increasing the amplitude or increasing the occurrence rate of brain waves in a specific frequency band); conversely, if the site is compared with the norm and it is “hyper-activity”, it is characterized by inhibiting the brainwaves in the brain area.

Brainwave assessment analysis from 1 to 19 electrode sites may be compared with the brainwave normative database of a single site, so it may be used through “surface brainwave training (Surface neurofeedback)”. After comparison, the percentage (% (or percentile rank, PR)) of the similarity between the collected brainwave EEG and the brainwave normative database may be obtained. Through the conversion of the Z standard score, the relative position of the brainwave pattern in the norm (too high or too low) may be known.

After the brainwaves are collected, the static brainwave results are compared with the database and the output is called “Quantitative electroencephalography (QEEG)”. But if it is used for real-time neurophysiological feedback of brainwaves (Real-time Neurofeedback), dynamic brainwaves are used for calculation and analysis. Taking a certain time window, as time shifts, the average number and standard deviation of the brainwaves recorded at each electrode site are calculated and analyzed in real time, and the 19-channel site may further analyze the activity of the brain network in real time. It provides feedback control, and then provides feedback on the user's brainwave status through auditory or visual means.

Therefore, the collected EEG brainwave data will be compared with the “health norm” and the “clinical norm”, and their corresponding similarity percentage values or percentiles will be compared. The corresponding similarity percentage value or percentile is a two-dimensional concept. That is to say, if the brainwave belongs to a healthy person, it should be compared with the brainwave norm of a healthy person and the percentage or percentile should have “high” similarity or correlation. The percentage or percentile of the comparison results of the same healthy person's brainwaves and the diseased brainwave norm should have a “low” degree of similarity or correlation.

Through the origin distribution of brain currents at 19 sites, it may be known whether the activity of specific brain areas or networks is too high or too low. The scalp surface (Surface) may be understood as 2D positioning, which may also monitor relatively shallow brain electrical activities; the neural network (Network) may be understood as 3D positioning, which may be traced to deeper brain electrical activities. Currently, 19-channel electrode sites are used for traceability and positioning. However, with the self-evolution learning of the calculation database, in the future, it may be possible to accurately predict brain area activity patterns using <19 sites (for example, a single site or a single-digit number of sites).

According to the above-mentioned method S100 of automatically screening neurofeedback training protocols and recommending results according to the present invention, after capturing the subject's brainwave data, a software interface is used to provide recommended training parameters (such as training parameter recommendations), the arrangement of strengths and weaknesses of the brain area network (such as 19-channel brainwave data), and digital therapy recommended training programs (such as training protocols and cognitive training) and related health education to achieve remote feedback and home brain training to improve cognitive abilities.

1. A method for automatically screening neurofeedback training protocols and recommending results, comprising:
capturing a biological data of a subject by a brainwave collection device, wherein the brainwave includes a brainwave collection device;
transmitting the biological data to a brainwave database of a remote cloud system through a network;
converting the biological data into a corresponding training parameter recommendation by the brainwave database; and
remotely feeding back the training parameter recommendation to a neurofeedback cognitive training module for the subject to perform cognitive training.

2. The method according to claim 1, wherein the home brainwave collection device is an electroencephalography (EEG) cap or a heart rate variability cap (HRV Cap).

3. The method according to claim 1, wherein the network is an ad-hoc Network.

4. The method according to claim 1, wherein the biological data is scalp electroencephalography (EEG) signals from one or more channels, wherein the training parameter recommendation is mainly converted into a standard score through at least one channel of the scalp electroencephalography (EEG) signals according to the calculation, and then presented in an order list according to a deviation mean.

5. The method according to claim 4, wherein the brainwave data includes amplitudes, frequencies, sites and pattern characteristics.

6. The method according to claim 1, wherein after the step of remotely feeding back the training parameter recommendation to a neurofeedback cognitive training module for the subject to perform cognitive training, the method further comprises providing an effect suggestion based on a result of the cognitive training, and transmitting the result of the cognitive training through the network back to the remote cloud system.

7. The method according to claim 1, wherein the neurofeedback cognitive training module includes a desktop computer, a notebook computer and/or a smart mobile device.

8. The method according to claim 1, wherein the training parameters are recommended to include a low-resolution electromagnetic tomography (LORETA) brain area/network training and a surface brainwave training.

9. The method according to claim 5, wherein a brain activity area is predicted back through the brainwave characteristics as the recommended training parameters based on the sites of the one or more channels.

10. The method according to claim 1, wherein the step of converting the biological data into a corresponding training parameter recommendation by the brainwave database further comprises recommending to refer to a priority order of execution of training after comparing surface brainwaves and brain area network according to a norm through a training protocol recommendation after the calculation; and remotely feeding back to a neurofeedback cognitive training module based on the training parameter recommendation for the subject to perform a cognitive training step of neurophysiological feedback, and then comparing the norm to evaluate whether brainwaves or brain areas are approaching balance to evaluate effectiveness recommendations.
